(19) United States
(12) Patent Application Publication
(10) Pub. No.: US 2011/0306894 A1
(43) Pub. Date: Dec. 15, 2011

(54) SYSTEMS, METHODS AND APPARATUS FOR ACQUIRING AND MANAGING PATIENT/SUBJECT DATA

(76) Inventors: Randol R. Spaulding, West Bend, WI (US); Brock E. Heinz, Grafton, WI (US); Paul B. Schultz, Grafton, WI (US); Jay W. Mason, Reno, NV (US)

(21) Appl. No.: 13/158,141

(22) Filed: Jun. 10, 2011

Related U.S. Application Data
(60) Provisional application No. 61/354,294, filed on Jun. 14, 2010.

(51) Int. Cl. A61B 5/0468; A61B 5/0432

(52) U.S. Cl. 600/515; 600/523

(57) ABSTRACT

An ECG acquisition device including a central processing unit, an input interface connected to the central processing unit for acquiring subject biometric data, a memory module connected to the central processing unit for storing the subject biometric data and ECG data, and a user interface connected to the central processing unit for initiating acquisition of the subject biometric and ECG data. The ECG acquisition device is connected to a site PC for downloading and managing the ECG and biometric data, which is transmitted to a Clinical Information System by the site PC.

### RELATED APPLICATIONS

The present patent application claims priority to U.S. provisional patent application 61/354,294 filed Jun. 14, 2010, the entire contents of which are incorporated herein by reference.

### BACKGROUND

The present invention relates to systems, methods and apparatus for acquiring and managing physiological and biometric data from a patient or living test subject.

### SUMMARY

In one embodiment, the invention provides an ECG acquisition device. The ECG acquisition device includes a central processing unit; an input interface connected to the central processing unit for acquiring subject biometric data; a memory module connected to the central processing unit for storing the subject biometric data; and a user interface connected to the central processing unit for initiating acquisition of the subject biometric data.

In another embodiment the invention provides a system for acquiring and storing data. The system includes a computer connectable to an ECG acquisition device. The ECG acquisition device includes inputs for acquiring subject biometric data, acquiring subject ECG data, and programmed instructions for communicating with the computer. The computer includes programmed instructions for receiving the subject biometric data and subject ECG data, verifying the subject biometric data and subject ECG data, storing the verified data and deleting the subject biometric data and the subject ECG data from the ECG acquisition device after storing the data.

In yet another embodiment, the invention provides a method of acquiring and managing data acquired from a subject using a data acquisition device connectable to a specially programmed computer. The method comprises acquiring biometric data from a subject and storing the biometric data on the acquisition device; acquiring ECG data from the subject, and storing the ECG data on the acquisition device; connecting the acquisition device to the specially programmed computer; transferring the data to the specially programmed computer; and deleting the data from the acquisition device.

Other aspects of the invention will become apparent by consideration of the detailed description and accompanying drawings.

### BRIEF DESCRIPTION OF THE DRAWINGS

FIG. 1 is a block diagram of a system embodying the invention.

FIG. 2 is a schematic block diagram of an acquisition device embodying the invention.

FIG. 3 is a schematic block diagram of a site personal computer (“PC”) including specially programmed software modules embodying the invention.

FIG. 4 is a flowchart of the process of using the acquisition device to acquire data from a test subject visit.

FIG. 5 is a flowchart illustrating the process of uploading ECG and biometric data of the test subject from the acquisition device to the site PC, and from the site PC to the Clinical Information System (“CIS”).

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings, whether direct or indirect. Also, electronic communications and notifications may be performed using any known means including direct connections, wireless connections, etc.

It should be noted that a plurality of hardware and software based devices, as well as a plurality of different structural components may be utilized to implement the invention. Furthermore, and as described in subsequent paragraphs, the specific configurations illustrated in the drawings are intended to exemplify embodiments of the invention and that other alternative configurations are possible. The terms “processor” “central processing unit” and “CPU” are interchangeable unless otherwise stated. Where the terms “processor” or “central processing unit” or “CPU” are used as identifying a unit performing specific functions, it should be understood that, unless otherwise stated, those functions can be carried out by a single processor, or multiple processors arranged in any form, including parallel processors, serial processors, tandem processors or cloud processing/cloud computing configurations.

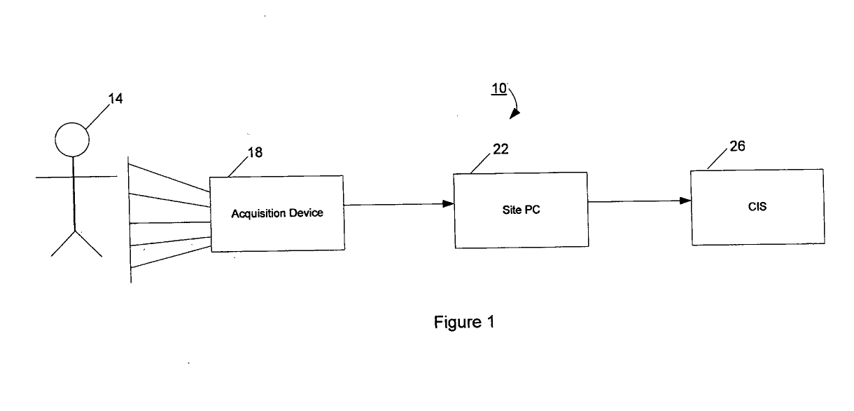

FIG. 1 illustrates a block diagram of a system 10 that acquires and manages physiological and biometric data of a patient or clinical trial test subject (hereinafter “subject”) 14. The system includes an acquisition device 18 connectable to an on-site personal computer (site PC) 22 that communicates with a remote clinical information system CIS 26.

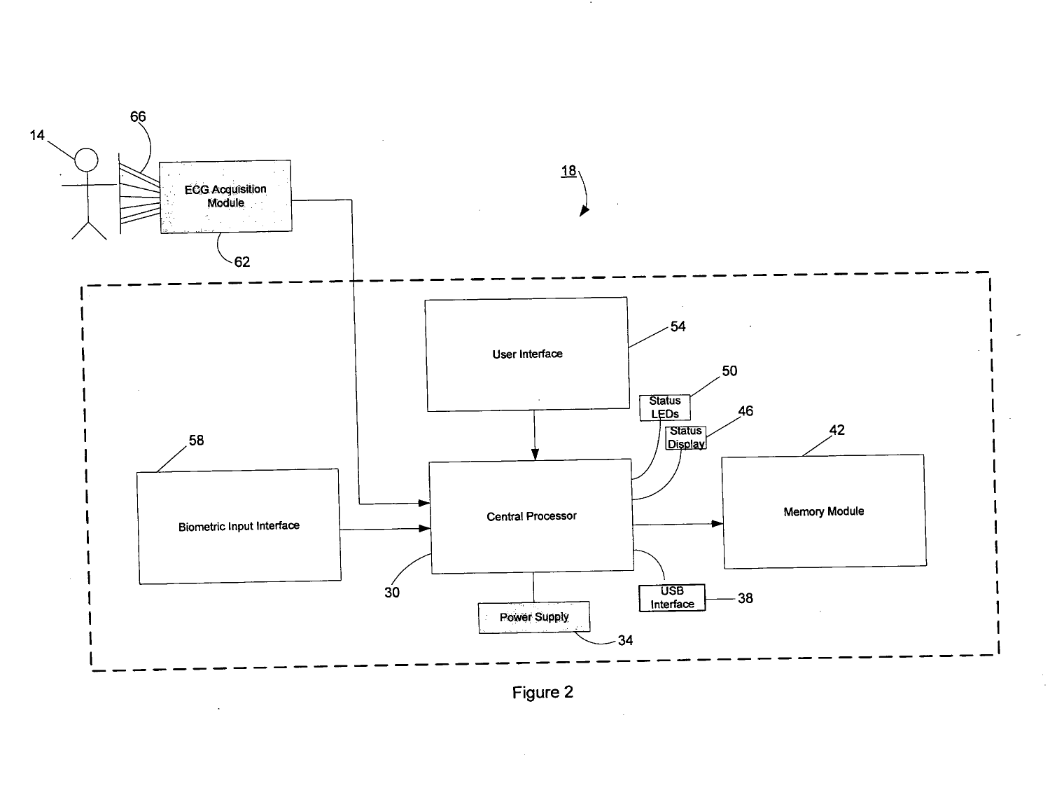

Shown in FIG. 2 is a schematic block diagram of the acquisition device 18. The acquisition device 18 includes a central processor 30. The central processor 30 is an AVR 8-Bit Microcontroller including non-transitory, programmed instructions to perform the operations of the acquisition device 18. The processor 30 includes 64 Kilobytes of flash memory and 8 Kilobytes random access memory, 4 Kilobytes of electronically erasable programmable read only memory (“EEPROM”) and a 12 bit A/D converter of 32 MHz. Other microprocessors, other memory modules, and other configurations of processors or memory modules can be substituted as those of skill in the art will appreciate. Each acquisition device 18 is programmed at the time of manufacture with a unique identifier. This identifier is included as a part of any data transmission sent from the acquisition devices 18. In particular, and as discussed in further detail below, the identifier is included as metadata in any transmission of ECG data and biometric data from the acquisition device 18.

The acquisition device 18 includes a power supply 34 to supply energy to the acquisition device 18. The power supply 34 includes a rechargeable lithium-ion polymer (“LiPo”) battery connectable to a 100 milliamp battery charger (not shown), an analog power regulator (not shown), and a digital power regulator (not shown), all of which are commonly known in the art. The LiPo battery is recharged via a USB connection when connected to the site PC.

The acquisition device 18 includes a universal serial bus (“USB”) input/output interface 38 connected to the processor 30. The USB interface 38 allows connection of the acquisition device 18 to an external device such as the site PC 22. Interfaces other than a USB interface 38 including wired or wireless connections of any sort may be substituted, as is apparent to those of skill in the art.

The acquisition device 18 includes a memory module 42 connected to the processor 30. The memory module 42 includes sufficient memory to store at least 300 seconds of subject ECG data and data representing a biometric of the subject. In the embodiment shown, the biometric data is thirty (“30”) seconds of recorded voice data. Other biometric signatures can be acquired as desired for the particular application.

The acquisition device 18 includes a status display 46 connected to the processor 30. The status display 46 is a conventional seven (“7”) segment light emitting diode (“LED”) array for displaying a single alpha-numeric character.

The acquisition device 18 includes a pair of status LEDs 50 connected to the central processor 30. In some embodiments, these LEDs could be internal to the device and used for development and/or troubleshooting purposes. The status LEDs 50 and status display 46 are used to communicate the device status including battery status, data storage, and data acquisition status. One status LED is red, and one status LED is green. Of course, other colors, numbers of LEDs, and configurations can be used to convey status of the acquisition device 18.

The acquisition device 18 includes a user interface 54 connected to the central processor 30. In the embodiment shown, the user interface 54 is a press-button switch (“button”). The button 54 allows a user to control functions of the acquisition device 18 such as recording biometric data, acquiring ECG data, and turning the acquisition device 18 “on” and “off.”

The acquisition device 18 includes a biometric input interface 58 connected to the central processor 30. The biometric input interface 58 is a microphone. The microphone reacts to the subject's voice and converts the voice to an electronic format for transmission to the central processor 30. Other biometric interface inputs can be used, especially where the acquired biometric is something other than a voice recording. For example, if the desired biometric data is a retinal scan, the biometric input interface 58 would be a retinal scanner. The biometric input interface 58 could also be a fingerprint or hand scanner, or include a chemical sensor for analyzing biological fluids or materials, such as saliva, DNA, etc.

The acquisition device 18 includes an ECG acquisition module 62 connected to the central processor 30. The ECG acquisition module 62 includes a conventional ECG patient cable removably connectable to the acquisition device 18 through a connector (not shown) on the acquisition device 18. As is commonly known in the art, the ECG patient cable includes a conventional set of ten (“10”) patient leads 66 that are connected to the patient in a known configuration to generate the classic twelve lead ECG. As is commonly known in the art, the ECG acquisition device also includes software (not shown) for reducing noise, baseline wander and/or offset of signals acquired from the patient.

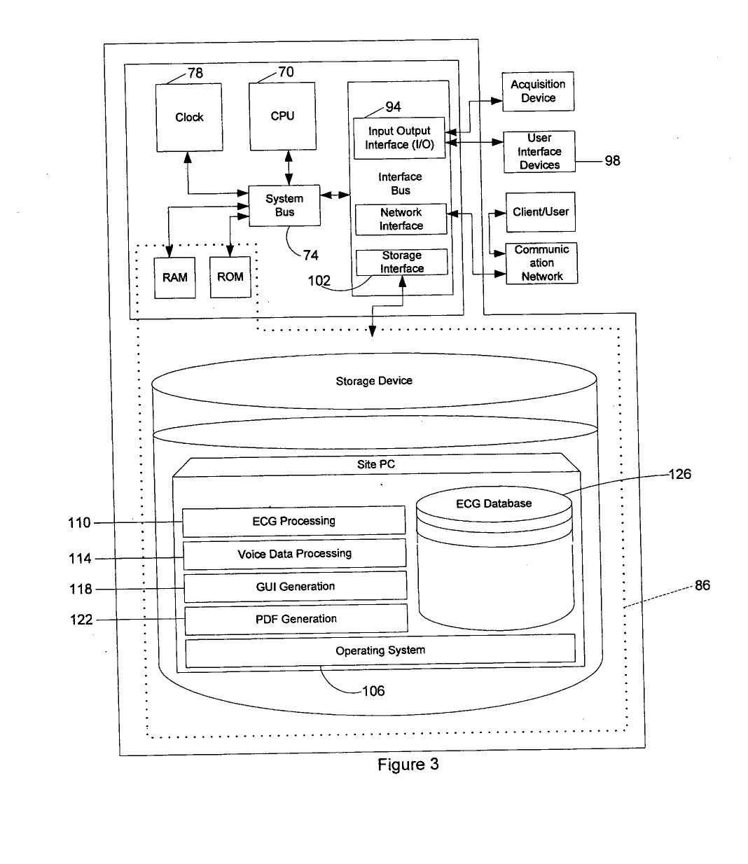

FIG. 3 is a schematic illustration of the site PC 22 of the system 10. The site PC 22 includes a CPU 70, a system bus 74, a clock 78, an interface bus 82 and a power supply (not shown). The CPU 70 is connected to the system bus 74 and includes and executes non-transitory programmed instructions stored in the CPU 70 or memory to operate the site PC 22. Other microprocessors or controllers can be used solely or in combination to perform the same functions as the CPU 70.

The system bus 74 transfers data between the various systems and modules of the site PC 22. The system bus 74 is also connected to the clock 78, and the interface bus 82. The system bus 74 transfers data and programmed instructions between the CPU 70 and memory 86. As is commonly understood by those of skill in the art, the clock 78 sets the speed at which programmed instructions are executed within the site PC 22.

The interface bus 82 includes a network interface 90. The network interface 90 is connectable to a communication network such as a LAN, WAN, the internet, or any other network allowing access to a remote device or computer. The connection can be achieved through any commonly used means, such as wired or wireless connections (including WiFi, wireless air card, satellite, or other means).

The interface bus 82 includes an input/output interface 94. The input/output interface 94 includes a plurality of ports (such as, for example, USB, BlueTooth, WiFi, etc.) that allow for connection between the site PC 22 and the acquisition device 18, as well as user interface devices 98, such as a mouse (or other point/click devices), keyboard, printer and/or a video display. In one form, the input/output ports (not shown) are preferably in standard universal serial bus “USB” configuration, and therefore allow for easy connection between the site PC 22 and the user input/output devices. The ports may include a separate video format connection for generating video on the video display. The interface bus 82 also includes a storage interface 102 connected to the memory 86. The storage interface 102 is a communication interface with the memory 86.

The site PC 22 also includes an operating system 106, a plurality of software modules (110, 114, 118, 122), and a local ECG database for site specific ECGs and subject demographics 126. The operating system 106 includes non-transitory program instructions for operating the site PC 22. The software modules include an ECG processing module 110, a voice scoring and visit correlation module 114, a graphical user interface (hereinafter “GUI”) generation module 118 and a PDF generation module 122, for generating ECG traces. The ECG processing module 110 includes non-transitory programmed instructions for processing ECG data for rendering. The ECG processing module 110 determines whether stored ECGs meet simple quality standards and also performs non interpretive ECG analysis for presentation of full disclosure data and best 10-second strips. The raw data file is retained locally in the Site PC until confirmation of the automatic transfer to the CIMS has been received. The ECG analysis services performed at the CIMS is based on study specific contracted services which can include basic ECG rhythm analysis, interval analysis, diagnostic analysis, and morphology analysis. The rhythm analysis includes generation of an abbreviated “Hotter” report providing a summary table showing the frequency of normal and abnormal beats, as well as the detection of abnormal beats and analysis of heart-rate-variability. The interval analysis calculates and provides measurements of beat parameters such as RR interval, PR interval, QRS interval, and QT interval. Summary statistics are also provided for each interval analyzed, including mean value, standard deviation, and minimum and maximum values. The diagnostic analysis also submits the ECG data to an AMPS CalECG program and the Glasgow Algorithm to (but not limited to) develop a synthesized ECG that is representative of the full set of ECG data and late potentials (SAECG) The morphology analysis extracts portions of the data and provides standard measurements of morphology such as T-wave amplitude, as well as more complex analyses generated by applying the Gaussian Mesa Function to model the T-wave.

The voice data processing module 114 includes non-transitory program instructions for analyzing the voice recording to determine whether the voice recording matches a prior recording for the test subject. In the embodiment shown, the voice data processing module 114 is the Vocalect software program developed and marketed by Voice Biometrics Group of Newtown, Pa. In another embodiment, the site PC 22 can transmit the voice recording to a remote computer storing the voice analysis software. The analysis is performed on the remote computer and the results are transmitted back to the site PC 22.

The GUI generation module 118 includes non-transitory program instructions for generating the various screens on the site PC 22 display, including the ECG viewer. The PDF generation module 122 includes non-transitory program instructions for generating the reports produced by the system. The ECG database 126 includes memory 86 for storing the results of the ECG analyses discussed above, the ECG data, biometric data, metadata and subject demographics associated with the ECG and biometric data.

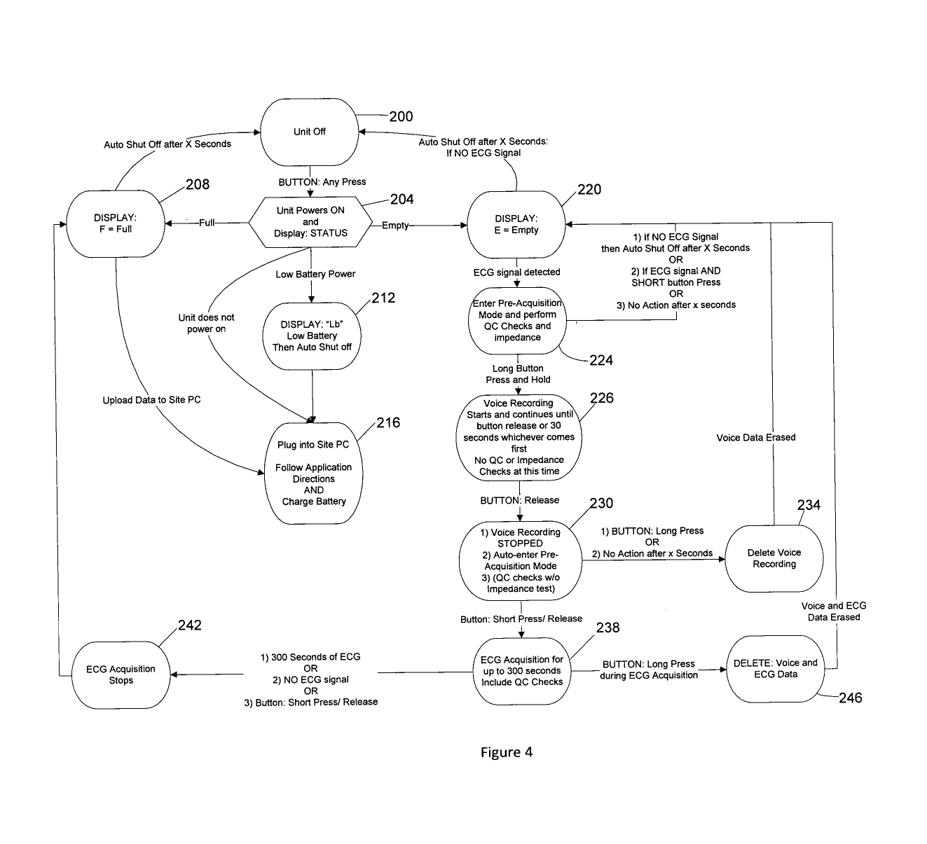

FIG. 4 is a flowchart of the process of using the acquisition device 18 to acquire data from a test subject 14 visit. The entry point 200 into the flowchart in FIG. 4 indicates that the acquisition device 18 is “off” and ready to record subject ECG and biometric data. In other words, the battery is sufficiently charged, and there is no ECG or biometric data currently stored in the memory 42 of the acquisition device 18. At 204, the user (not shown) turns the acquisition device 18 “on” by depressing the push button 54 and does a visual check of the status indicators to confirm that the acquisition device 18 is ready. If the device memory 42 is full, as indicated at 208, the unit will indicate this fact to the user and turn itself “off”. It will continue to do so until connected to the site PC 22, downloads its data thereto, and the memory is cleared as shown at 216. The unit will also automatically turn itself “off” after a predetermined period, e.g., several seconds, if no other buttons are pressed, or if there is insufficient battery life to operate the unit as detected at 212. If the acquisition device 18 is not ready because the battery is depleted, the clinician can recharge the battery by connecting the acquisition device 18 to the site PC 22 for a period of time as shown at 216.

At 220, if the user sees the device memory is empty, and the battery is charged, the user connects the ECG acquisition module 62 to the acquisition device 18, connects the leads of the ECG cable to electrodes (not shown) attached to the test subject 14. At 224, the acquisition module detects the ECG signal and enters a pre-acquisition mode in which it performs “quality checks” on the connection to the test subject. For example, the device 18 tests the impedance of the connections with the patient to determine whether all leads are connected to the test subject and whether they are correctly connected, as well as detects and accounts for baseline wander, noise, offset, muscle artifact and other aspects of the ECG. At 226, the user prompts the test subject 14 to speak a prepared statement for a predetermined period, e.g., thirty (“30”) seconds. The user holds the push button 54 to record the voice of the test subject for use as a biometric identifier. The recording stops when the button 54 is released, or after 30 seconds has transpired, as indicated at 230.

While the acquisition device is sampling ECG signal for quality assessment, the user may initiate re-recording the voice data by pressing the button 54 and holding the button 54 for a predetermined period, e.g., more than one second (at 234). If the user depresses the button 54 for less than the predetermined period, the acquisition device 18 begins recording the ECG of the test subject 14 (at 238). The recording of ECG data continues for a predetermined period, e.g., until 300 seconds of ECG data is acquired and then stops at 242. During this time, the user may stop ECG acquisition by pressing the button 54 for given period of time, e.g., less than one (“1”) second. The user may then re-initiate ECG acquisition. The user may also press the button for greater than a predetermined period (as shown at 246) to stop ECG recording, erase voice and ECG data and reinitiate the process. If acquisition is not suspended before the full 300 seconds of data is acquired, then the device indicator changes to indicate the device storage is “full”, and the device turns “off”. If anyone attempts to turn the device 18 “on”, the device 18 will turn “on” briefly, displaying indicators that the device storage is full, and then automatically turn itself “off”, as shown at 208.

The patient 14 is then disconnected from the acquisition device 18. All ECG and biometric data is retained in the acquisition device 18 until imported to the site PC 22 (at 216), whereupon the data is deleted from the acquisition device 18. In this way, duplicate data is never imported to the site PC 22, and the storage memories of the acquisition devices 18 are “cleaned” in preparation for the next use. Moreover, when the ECG data and biometric data is transmitted to the site PC 22, it is combined with metadata including the unique acquisition device identifier, as well as a checksum used to verify the integrity of the data transmission to the site PC 22, and error detection codes, if any.

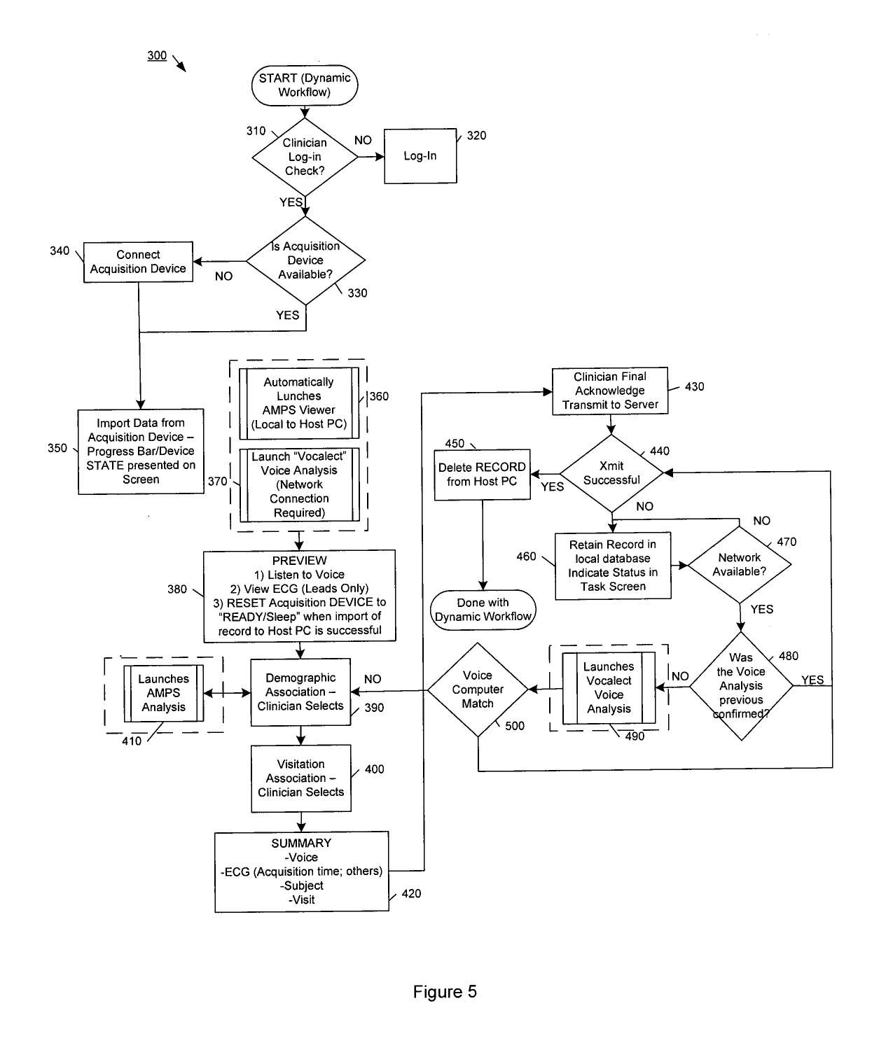

FIG. 5 is a flowchart illustrating the process 300 of uploading ECG and biometric data of the test subject from the acquisition device 18 to the site PC 22. At steps 310 and 320 a user logs onto the site PC 22 and enters the test subject's demographic information (this test subject could be one participating in a current study being implemented using the system, such as a human drug trial). With each system login/logout, a system related audit trail is generated/updated that identifies the time and date of the activity, as well as the identity of the user performing the activity. This audit trail is in both electronic and human readable form, and is stored in the site PC 22. At step 330, the user evaluates, based on status display 46 whether the acquisition device 18 has data stored on the device and is ready for uploading. If there is data on the acquisition device 18, the user connects (340) the acquisition device 18 to the site PC 22. When this occurs, the site PC 22 application is launched by the site PC 22 operating system 106. While connected to the site PC 22, the acquisition device 18 battery is charged.

At step 350, the central processor 30 of the acquisition device 18 generates an electronic record which contains the biometric data, ECG data, and the metadata (including the unique identifier of the acquisition device 18). The electronic record is imported to the site PC 22.

At steps 360 and 370, the site PC 22 automatically launches the ECG processing module 110 and the voice data processing module 114. The ECG processing module 110 allows the user to view and print the unconfirmed ECG, and perform quality checks to confirm the ECG meets quality standards. The voice data processing module 114 performs voice analysis on the biometric data. At step 380, the clinician will be prompted by an application on the site PC 22 to preview the acquired ECG data, listen to the voice recording, if desired, and reset the acquisition device 18 when the import of data to the site PC 22 is successful.

If this is the first visit for the test subject, the user enters the test subject's demographic information (e.g., name, sex, age, etc.) (at 390) and correlates the demographic data with the current ECG data, biometric data and metadata. If this is the second or later visit of the test subject, the user confirms the ECG data, biometric data and metadata with the subject's existing demographics and test subject visit information (400). The site PC 22 generates a summary report of the test subject's visit, including the voice recording (preferably stored electronically as a .wav file), ECG data (with acquisition time and other site visit data), and metadata. The user acknowledges the summary report and transmission of data to the site PC 22. If there is no acknowledgement by the user (meaning the clinician determined the acquired data was unable to be paired) then an error notice is generated on the display. The error notice includes information and instructions as to the possible problem and how to fix the problem. For example, in some cases a subject may be involved in a study that requires the subject to come back to a testing site on numerous occasions. However, if the test subject visits too frequently, an error notice may be generated.

At step 410, the site PC 22 launches the ECG processing module 110 (“the AMPS Analysis Tool”). The ECG data is automatically processed once the import has been successfully completed. As discussed above, the ECG processing module 110 performs analysis on the ECG data.

The user may review the rendering of the ECG data. The correlation of the ECG data to the demographics and subject visit data will be presented on a demographics/visit form stored electronically in the site PC 22 as a summary (420). In one embodiment, the PDF generation module 122, part of the ECG viewer application, can generate a PDF report of the summary that includes subject demographics, date and time of the ECG reading, visit name, test sponsor name, protocol name, device identifier, investigator or user name, and other information desired.

At step 430, the correlation of the ECG data to the demographics data and visit information are confirmed and the record (e.g., ECG metadata) is automatically uploaded via the communication network to the CIS 26 for further processing. At step 440, a confirmation of the successful transmission of the record as having been received by the CIS 26 is transmitted to the site PC 22 by the CIS 26. In one form, the site PC 22 deletes the record from the Site PC 22 after receiving an automatic confirmation that the data was successfully transmitted to the CIS 26 (at step 450).

At step 460, if the transmission is not successful the system retains the imported data and record in the local ECG database 126. At step 470 the site PC 22 verifies whether a network connection is available. If there is no network available, the system continues to retain the imported data and summary from the local database and checks for an available network until one is verified as available. At step 480, the CIS 26 analyzes the voice data and generates a score indicating the strength of correlation to a previous voice recording for a subject.

At step 490, the voice analysis is performed by the CIS 26. When a positive match has been identified using the score generated at step 480, an indication is sent to the site PC 22 for confirmation of the match by the clinician. If a match cannot be determined, an indication is sent to the site PC 22 prompting the clinician to either enter demographics for a new subject as would be the case for an initial visit; or select demographics from a list of subjects resident in the site PC subject database.

Various features and advantages of the invention are set forth in the following claims.

What is claimed is:

1. An ECG acquisition device comprising:
a central processing unit;
an input interface connected to the central processing unit for acquiring subject biometric data;
a memory module connected to the central processing unit for storing the subject biometric data; and
a user interface connected to the central processing unit for initiating acquisition of the subject biometric data.

2. An ECG acquisition device as set forth in claim 1, and further comprising an ECG acquisition module 62 for acquiring subject ECG data.

3. An ECG acquisition device as set forth in claim 2, wherein the central processing unit prevents acquisition of subject ECG data until the subject biometric data has been acquired and stored in the ECG acquisition device.

4. An ECG acquisition device as set forth in claim 2, where the central processing unit includes programmed instructions to check the quality of the ECG subject data and prevent storage of the ECG subject data until the quality meets acceptability standards.

5. An ECG acquisition as set forth in claim 1, wherein the ECG acquisition device is a portable, hand-held device.

6. An ECG acquisition device as set forth in claim 1, wherein the user interface for initiating acquisition of the subject biometric data also allows initiation of acquisition of the subject ECG data.

7. An ECG acquisition device as set forth in claim 1, and further comprising a USB interface connected to the central processing unit.

8. An ECG acquisition device as set forth in claim 1, and further comprising a status indicator connected to and controlled by the central processing unit.

9. An ECG acquisition device as set forth in claim 1, wherein the central processing unit includes programmed instructions creating a device identifier unique to each ECG acquisition device.

10. An ECG acquisition device set forth in claim 1, wherein the acquisition device includes programmed instructions to perform a preliminary noise assessment on the subject ECG data to evaluate the quality of the connection between the subject and the acquisition device.

11. An ECG acquisition device set forth in claim 1 and further comprising programmed instructions to create a data record including ECG acquisition device identification information and subject ECG data to a data record.

12. An ECG acquisition device comprising:
a central processing unit;
a USB interface connected to the central processing unit;
an input interface connected to the central processing unit for acquiring subject biometric data;
a memory module connected to the central processing unit for storing the subject biometric data;
a user interface connected to the central processing unit for initiating acquisition of the subject biometric data;
an ECG acquisition module for acquiring subject ECG data; and
wherein the central processing unit includes programmed instructions to prevent acquisition of subject ECG data until the subject biometric data has been acquired and stored in the ECG acquisition device, check the quality of the ECG subject data, and prevent storage of the ECG subject data until the quality meets acceptability standards.

13. A system for acquiring and storing data, the system comprising:
a computer connectable to an ECG acquisition device, the ECG acquisition device including inputs for acquiring subject biometric data and acquiring subject ECG data, the computer including programmed instructions for receiving the subject biometric data and subject ECG data, verifying the subject biometric data and subject ECG data, storing the verified data and deleting the subject biometric data and the subject ECG data from the ECG acquisition device after storing the data.

14. A system for acquiring and storing data as set forth in claim 13, wherein the computer further comprises means for communicating with a remote clinical information system (CIS) and programmed instructions for contacting the CIS, uploading the data to the CIS and deleting the data from computer when transfer of the data to the CIS has been confirmed.

15. A system for acquiring and storing data as set forth in claim 13, wherein the computer includes programmed instructions to generate an audit trail of changes and edits made to an electronic record of stored subject ECG data and generate an audit trail of activities related to the system such as a track and log of system logins and logouts.

16. A system for acquiring and storing data as set forth in claim 13, wherein the acquisition device includes programmed instructions to perform a preliminary noise assessment to the subject ECG data to evaluate the quality of the connection between the subject and the acquisition device.

17. A system for acquiring and storing data as set forth in claim 16, wherein the acquisition device includes programmed instructions to perform a lead off detection check, baseline wander check, and muscle artifact check to the subject ECG data to detect error in the subject ECG data.

18. A system for acquiring and storing data as set forth in claim 13, wherein the acquisition device includes programmed instructions to transmit ECG acquisition device identification information with the subject ECG data.

19. A system for acquiring and storing data as set forth in claim 13, wherein the computer includes programmed instructions to generate a report including a date and time of an ECG acquisition and an acquisition device unique identifier.

20. A system for acquiring and storing data as set forth in claim 13, wherein the computer includes independent software modules programmed to perform analysis on the subject ECG data, the independent software modules include but are not limited to a rhythm diagnostics module programmed to generate a report that provides a summary table with the frequency of normal and abnormal heartbeats, an interval analysis module capable to execute on a predetermined-time interval ECG reading, a diagnostic analysis module capable to generate a diagnosis for the subject ECG data that has been extracted and parsed, and a morphology module configured to take the subject ECG extractions that were previously measured and average the subject ECG extractions out with a subject ECG data reading measured according to morphology standards.

21. A system for acquiring and storing data, the system comprising:
a computer connectable to an ECG acquisition device, the ECG acquisition device including inputs for acquiring subject biometric data and acquiring subject ECG data, programmed instructions to perform a preliminary noise assessment to the subject ECG data to evaluate the quality of the connection between the subject and the acquisition device, a lead off detection check, a baseline wander check, a muscle artifact check to detect error in the subject ECG data, and transmit ECG acquisition device identification information with the subject ECG data to a data record, the computer including programmed instructions for receiving the paired subject biometric data and subject ECG data, verifying the paired subject biometric data and subject ECG data, storing the verified data, deleting the subject biometric data and the subject ECG data from the ECG acquisition device after storing the data, generating an audit trail of changes and edits made to an electronic record of stored subject ECG data, generate an audit trail of activities related to the system including a log of system logins and logouts, generating a report including a date and time of an ECG acquisition and an acquisition device unique identifier and a plurality of software modules programmed to perform analysis on the subject ECG data, the independent software modules include but are not limited to a rhythm diagnostics module programmed to generate a report that provides a summary table with the frequency of normal and abnormal heartbeats, an interval analysis module capable to execute on a predetermined-time interval ECG reading, a diagnostic analysis module capable to generate a diagnosis for the subject ECG data that has been extracted and parsed, and a morphology module configured to take the subject ECG extractions that were previously measured and average the subject ECG extractions out with a subject ECG data reading measured according to morphology standards; and
means for communicating with a remote clinical information system (CIS) and programmed instructions for contacting the CIS, uploading the data to the CIS and deleting the data from the computer from the computer when transfer of the data to the CIS has been confirmed.

22. A method of acquiring and managing data acquired from a subject using a data acquisition device connectable to a specially programmed computer, the method comprising:
acquiring biometric data from a subject and storing the biometric data on the acquisition device;
acquiring ECG data from the subject and storing the ECG data on the acquisition device in association with the biometric data;
connecting the acquisition device to the specially programmed computer;
transferring the data to the specially programmed computer; and
deleting the data from the acquisition device.

23. A method of acquiring and managing data as set forth in claim 22, and further comprising verifying and analyzing the data using the specially programmed computer;
transmitting the data to a clinical information system;
verifying the transfer; and
deleting the data from the specially programmed computer only when the transfer has been verified.

24. A method as set forth in claim 22, and further comprising analyzing the ECG data using independent software modules;
a rhythm diagnostics module generating a report that provides a summary table with the frequency of normal and abnormal heartbeats;
an interval analysis module executing being executed on a predetermined-time interval ECG reading;
a diagnostic analysis module generating a diagnosis for the subject ECG data that has been extracted and parsed; and
a morphology module taking the subject ECG extractions that were previously measured and averaging the subject ECG extractions out with a subject ECG data reading measured according to morphology standards.

25. A method as set forth in claim 22, and further comprising verifying the ECG data using the acquisition device;
performing a preliminary noise assessment to the subject ECG data to evaluate the quality of the connection between the subject and the acquisition device.

26. A method as set forth in claim 22, and further comprising tracking and logging usage of the system using the specially programmed computer;
generating an audit trail of changes and edits made to an electronic record of stored subject ECG data and generating an audit trail of activities related to the system such as a track and log of system logins and logouts.

27. A method as set forth in claim 22, and further comprising verifying ECG data using the acquisition device;
performing a lead off detection check, baseline wander check, and muscle artifact check to detect error in the subject ECG data.

28. A method as set forth in claim 22, and further comprising storing identifying information for the ECG acquisition device using the specifically programmed computer;
transmitting the ECG acquisition device identification information with the subject ECG data to a data record.

29. A method as set forth in claim 22, and further comprising storing the ECG data using the specifically programmed computer;
generating a report including a date and time of an ECG acquisition and an acquisition device unique identifier.

30. A method of acquiring and managing data acquired from a subject using a data acquisition device connectable to a specially programmed computer, the method comprising:
acquiring biometric data from a subject and storing the biometric data on the acquisition device;
performing a preliminary noise assessment to the subject ECG data to evaluate the quality of the connection between the subject and the acquisition device;
performing a lead off detection check, baseline wander check, and muscle artifact check to detect error in the subject ECG data;
acquiring ECG data from the subject, and storing the ECG data on the acquisition device in association with the biometric data;
connecting the acquisition device to the specially programmed computer;
transferring the data to the specially programmed computer;
verifying the transfer between the acquisition device and the specifically programmed computer;
deleting the data from the acquisition device;
generating an audit trail of changes and edits made to an electronic record of stored subject ECG data and generating an audit trail of activities related to the system such as a track and log of system logins and logouts;
transmitting the ECG acquisition device identification information with the subject ECG data to a data record;
transmitting the data to a clinical information system;
verifying the transfer;
deleting the data from the specially programmed computer only when the transfer has been verified; and
wherein further analysis of the ECG data is performed by programming modules, including a rhythm diagnostics module generating a report that provides a summary table with the frequency of normal and abnormal heartbeats, an interval analysis module executing being executed on a predetermined-time interval ECG reading, a diagnostic analysis module generating a diagnosis for the subject ECG data that has been extracted and parsed; and a morphology module taking the subject ECG extractions that were previously measured and averaging the subject ECG extractions out with a subject ECG data reading measured according to morphology standards.
